(19) United States
(12) Patent Application Publication
(10) Pub. No.: US 2005/0192447 A1
(43) Pub. Date: Sep. 1, 2005

(54) Process for preparing alkylene oxide

(76) Inventor: Gerard Du Cauze De Nazelle, Jurong Island (SG)

Correspondence Address: Jennifer D. Adamson;Shell Oil Company, Legal - Intellectual Property, P.O. Box 2463, Houston, TX, 77252-2463, US

(21) Appl. No.: 11/049,839

(22) Filed: Feb. 3, 2005

(30) Foreign Application Priority Data
Feb. 2, 2004 (EP) 04250625.3

(51) Int. Cl. C07D 301/19; C07C 409/00

(52) U.S. Cl. 549/529; 568/568

(57) ABSTRACT

The invention relates to a process for the preparation of alkylene oxide involving: (a) oxidizing an organic compound to obtain a hydroperoxide containing stream, (b) washing the hydroperoxide stream with a basic aqueous solution, (c) washing the hydroperoxide stream of step (b) with water, (d) optionally subjecting the hydroperoxide stream obtained in step (c) to distillation, (e) contacting at least part of the hydroperoxide stream obtained in step (c) and/or (d) with alkene and a heterogeneous catalyst to obtain a reaction mixture containing a hydroxyl containing compound and alkylene oxide, and (f) separating at least part of the alkylene oxide from the reaction mixture, in which process the alkene added in step (e) has a temperature of from 60 to 120° C. while the temperature of the hydroperoxide stream contacted with the alkene is similar to the temperature of the hydroperoxide stream obtained in step (c) and/or (d).

### CLAIM TO PRIORITY

This application claims the benefit of European Application 04250625.3 filed Feb. 5, 2004.

### FIELD OF THE INVENTION

The present invention relates to a process for the preparation of an alkylene oxide.

### BACKGROUND OF THE INVENTION

Processes for preparing propylene oxide employing organic hydroperoxides are known in the art. As described in U.S. Pat. No. 5,883,268, such a process conventionally comprises peroxidation of ethylbenzene, followed by contacting the peroxidation reaction product with aqueous base in amount sufficient to neutralize acidic components thereof and separating the resulting mixture into an aqueous stream and a deacidified organic stream. The base contaminated, deacidified hydroperoxide stream is washed with water. The product obtained can be used in the catalytic epoxidation of propene to form propylene oxide using a solid heterogeneous titanium containing catalyst.

Although the epoxidation reaction is exothermic, the temperature of the mixture of hydroperoxide and alkene is usually increased before use in the epoxidation process. This ensures best use of the catalyst employed. However, it was found that the heat exchanger used for increasing the temperature of the mixture, fouled quickly when contacted with the mixture of hydroperoxide and alkene. It is disadvantageous if the temperature of the mixture of hydroperoxide and alkene cannot be increased as this makes that deactivation of the catalyst cannot be compensated for. This results in more frequent replacement of the epoxidation catalyst.

### SUMMARY OF THE INVENTION

The present invention is directed to a process for the preparation of alkylene oxide which process comprises:
(a) oxidizing an organic compound to obtain a hydroperoxide containing stream,
(b) washing the hydroperoxide stream with a basic aqueous solution,
(c) washing the hydroperoxide stream of step (b) with water,
(d) optionally subjecting the hydroperoxide stream obtained in step (c) to distillation,
(e) contacting at least part of the hydroperoxide stream obtained in step (c) and/or (d) with alkene and a heterogeneous catalyst to obtain a reaction mixture containing a hydroxyl containing compound and alkylene oxide, and
(f) separating at least part of the alkylene oxide from the reaction mixture,
in which process the alkene added in step (e) has a temperature of from 60° C. to 120° C. while the temperature of the hydroperoxide stream contacted with the alkene is similar to the temperature of the hydroperoxide stream obtained in step (c) and/or (d).

### DETAILED DISCUSSION OF THE INVENTION

It has now been found that the temperature of the reaction mixture to be contacted with the epoxidation catalyst may be heated to a relatively high temperature if a specific set-up is used. In this specific set-up, the temperature of the mixture of alkene and hydroperoxide is increased by increasing the temperature of the alkene only. Heating the alkene to a relatively high temperature does not cause fouling of the heat exchanger. Without wishing to be bound to any theory, it is thought that the fouling of the heat exchangers when heating a mixture of alkene and a hydroperoxide stream is caused by salts which are present in the hydroperoxide stream depositing on the surface of the heat exchanger when the hydroperoxide stream is mixed with the alkene.

In step (a) of the present invention, an organic compound is oxidized. The organic compound preferably is an alkylaryl compound, which is an alkyl substituted aromatic compound. The compounds which are most preferably used in the process of the present invention are benzene compounds containing at least 1 alkyl substituent which alkyl substituent contains from 1 to 10 carbon atoms, preferably from 2 to 8 carbon atoms. Preferably, the benzene compound contains on average from 1 to 2 constituents. The alkylaryl compounds most frequently encountered are ethylbenzene and cumene. In that case, the hydroperoxide compounds formed are ethylbenzene hydroperoxide and cumene hydroperoxide.

The oxidation of the alkylaryl compound may be carried out by any suitable process known in the art. The oxidation may be carried out in the liquid phase in the presence of a diluent. This diluent is preferably a compound which is liquid under the reaction conditions and does not react with the starting materials and product obtained. However, the diluent may also be a compound necessarily present during the reaction. For example, if the alkylaryl is ethylbenzene, the diluent may be ethylbenzene as well and if the alkylaryl is cumene the diluent may be cumene as well.

Other than the desired hydroperoxide compound, a range of contaminants may be created during the oxidation of organic compounds. Although most of these are present in small amounts, it has been found that the presence of compounds such as organic acids may cause problems in subsequent use of the hydroperoxide containing stream. As described in U.S. Pat. No. 5,883,268, which is herein incorporated by reference, the amount of contaminants may be reduced by contacting the hydroperoxide stream with a basic aqueous solution. Aqueous bases which may be used in the process include sodium and/or potassium containing bases such as sodium hydroxide, potassium hydroxide, sodium carbonate and potassium carbonate. Most preferably, the basic aqueous solution used in the present invention is an aqueous solution of sodium hydroxide. Preferably, the washing with a basic aqueous solution is carried out at a temperature of between 0° C. and 150° C., more preferably of between 20° C. and 100° C.

The washing of step (b) comprises both contacting with the basic aqueous solution and separating into a hydrocarbonaceous phase and an aqueous phase. A preferred separation method comprises allowing the hydro-carbonaceous phase and aqueous phase to settle in a settling vessel and subsequently separating a hydrocarbonaceous phase from an aqueous phase. The hydrocarbonaceous phase containing hydroperoxide may subsequently be sent to a coalescer where further aqueous phase is removed. Preferably, the separation step is carried out at a temperature between 0° C. and 150° C., more preferably between 20° C. and 100° C.

The hydroperoxide containing stream which has been treated with basic aqueous solution may be contacted with alkene without further treatment. However, contact with the basic aqueous solution introduces alkali metal into the hydroperoxide containing reaction product. Therefore, the hydroperoxide stream is subsequently washed with water.

The water wash of step (c) may be carried out in any way known to one skilled in the art. The water which may be used may contain contaminants, such as organic compounds. Such contaminants may have been introduced by the recycle of at least part of the wash water, either to the same wash step or to another wash step. The water may be fresh water only, it may be a combination of fresh water containing substantially no contaminants and one or more different waste water streams, or it may consist only of different kinds of waste water streams or it may consist of a single type of waste water.

The exact conditions under which the water wash is carried out depend on further circumstances. Preferably, the water wash is carried out at a temperature between 0° C. and 150° C., more preferably between 20° C. and 100° C. Separating the hydrocarbonaceous phase and the aqueous phase may subsequently be carried out in any way known to one skilled in the art. A preferred separation method comprises allowing the hydrocarbonaceous phase and aqueous phase to settle in a settling vessel and subsequently separating a hydrocarbonaceous phase from an aqueous phase. The hydrocarbonaceous phase containing hydroperoxide may subsequently be sent to a coalescer where further aqueous phase is removed. Preferably, the separation step is carried out at a temperature between 0° C. and 150° C., more preferably between 20° C. and 100° C.

The water wash may be carried out once or it may be repeated several times.

The reaction product of step (c) may be sent to step (e) as such. However, it is preferred to remove light compounds. Such light compounds may be unconverted organic compounds, water and contaminants. The light components may be easily removed by subjecting the reaction product of step (c) to distillation, preferably distillation at reduced pressure. A distillation which is especially suitable is so-called flash distillation, which comprises distillation at very low pressure. Such flash distillation is efficient in removing light compounds such as oxygen and light acids formed during the oxidation.

In process step (e), at least part of the hydro-peroxide stream obtained in step (c) and/or (d) is contacted with alkene in the presence of a heterogeneous catalyst to obtain a reaction mixture containing a hydroxyl containing compound and alkylene oxide. The alkene preferably is propene, which leads to propylene oxide as product. The hydroperoxide stream added to step (e) has a temperature which is similar to the temperature of the hydroperoxide stream obtained in step (c) and/or (d). Although it may sometimes be advantageous to slightly increase the temperature of the hydroperoxide stream, if, for example, the stream is obtained directly from step (c), it is preferred that the temperature of the hydroperoxide stream not be increased more than 10° C., more specifically not more than 5° C. Preferably, the temperature is not increased.

The alkene added in step (e) has a temperature of from 60° C. to 120° C. before contacting the hydroperoxide stream. This desired temperature may be obtained by heating the alkene. The alkene generally has a temperature of from 40° C. to less than 60° C. before heating, more specifically of from 45° C. to 55° C. Usually, the alkene will be heated using a heat exchanger. The stream against which the heat is exchanged may be any stream having the right temperature. Generally, steam will be used for heating the alkene. Preferably, the alkene to be introduced in step (e) has a temperature of at least 70° C., more preferably more than 75° C., more preferably more than 80° C. The alkene preferably has a temperature of at most 115° C., more specifically at most 110° C., more specifically at most 105° C., most specifically at most 100° C. Preferably, the temperature of the alkene added in step (e) is from 70° C. to 110° C. before contact with the hydroperoxide stream.

The specific temperature of the hydroperoxide stream and of the alkene depend on the circumstances such as amount of catalyst available for the reaction, the activity of the catalyst in general, the degree to which the catalyst has become deactivated and the molar ratio of alkene to hydroperoxide.

Generally, the hydroperoxide stream added in step (e) will have a temperature of from 70° C. to 120° C. before contact with the alkene. More specifically, the hydroperoxide stream added in step (e) will generally have a temperature of from 80° C. to 110° C. Preferably, the temperature of the hydroperoxide stream is at least 85° C., more specifically at least 90° C. The temperature of the hydroperoxide stream preferably is at most 105° C., most preferably at most 100° C.

With the process of the present invention, it is now possible to perform the epoxidation reaction of step (e) continuously at a higher temperature than was previously possible in a commercial unit. The hydroperoxide and the alkene may be contacted continuously with the catalyst at a temperature of more than 85° C. This temperature is the temperature of the mixture of hydroperoxide and the alkene when contacted with the catalyst for the first time. The temperature may be increased to at least 90° C. The temperature will generally be at most 115° C., more specifically at most 100° C. However, the exact temperatures depend on the catalyst which is used as mentioned hereinabove.

A heterogeneous catalyst which may suitably be used in step (e) is a titanium containing catalyst. A preferred catalyst contains titanium on silica and/or silicate. A preferred catalyst is described in EP-345856. The reaction generally proceeds at moderate temperatures and pressures, in particular at temperatures in the range of from 0° C. to 200° C., preferably in the range from 25° C. to 200° C. The precise pressure is not critical as long as it suffices to maintain the reaction mixture as a liquid or as a mixture of vapor and liquid. Atmospheric pressure may be satisfactory. In general, pressures may be in the range of from 1 to 100×10⁵ N/m².

The alkylene oxide may be separated from the reaction mixture obtained in step (e) in any way known to be suitable to one skilled in the art. The liquid reaction product may be worked up by fractional distillation, selective extraction and/or filtration. Any solvent, unreacted olefin and/or hydroperoxide may be recycled for further utilization. Preferably, in step (f) the alkylene oxide is separated by distillation from the reaction mixture.

The hydroxyl containing compounds obtained in the process may be dehydrated in the presence of a catalyst to obtain styrene and water. Processes which may be used for this step have been described in WO 99/42425 and WO 99/42426. However, any suitable process known to someone skilled in the art may in principle be used.

The invention is further illustrated by the following examples without limiting the scope of the invention to these particular embodiments.

### COMPARATIVE EXAMPLE 1

In a reactor, air was blown through ethylbenzene. The product obtained contained ethylbenzene hydroperoxide. This product was contacted with a solution containing 0.5% wt sodium hydroxide in water and mixed at a temperature of 60° C. The weight ratio of product containing ethylbenzene hydroperoxide to sodium hydroxide containing solution was 4.5:1 (wt:wt). The neutralized mixture obtained was sent to a settling vessel where a neutralized hydrocarbonaceous phase containing ethylbenzene hydroperoxide was separated from an aqueous phase.

The neutralized hydrocarbonaceous phase containing ethylbenzene hydroperoxide was sent to a coalescer where further aqueous phase was removed. Subsequently, the neutralized hydrocarbonaceous phase containing ethylbenzene hydroperoxide was washed by mixing with water, separating the mixture obtained in a settling vessel into an aqueous phase and a hydrocarbonaceous phase, and subsequently separating the hydrocarbonaceous phase obtained from the settling vessel with a coalescer. The hydrocarbonaceous phase obtained in the coalescer contained ethylbenzene hydroperoxide, ethylbenzene, water and contaminants. This hydrocarbonaceous phase was distilled. The distillate contained ethyl benzene, water and contaminants. The bottom product contained ethylbenzene hydroperoxide and ethylbenzene.

The ethylbenzene hydroperoxide product contained between 30 and 40% wt of ethylbenzene hydroperoxide in ethylbenzene. A feed was obtained by mixing propene having a temperature of about 50° C. and the ethylbenzene hydroperoxide product having a temperature of about 97° C. in such amounts that the molar ratio of propene to ethylbenzene hydroperoxide was about 6. The feed obtained was heated in a heat-exchanger in which the heat was provided by steam having a temperature of about 160° C. and a pressure of about 5 bar (5×10⁵ N/m²).

At the start of operation, the feed was heated to about 95° C. After several weeks of unchanged operation, the feed leaving the heat exchanger had a temperature of about 85° C. This reduction in temperature attained shows substantial fouling of the heat exchanger.

### EXAMPLE 1

The process set-up of Comparative Example 1 was changed such that the propene having a temperature of about 50° C. was heated alone in the heat exchanger to a temperature of about 85° C. The heated propene was subsequently combined with the ethylbenzene hydroperoxide product having a temperature of about 97° C.

The combined heated propene and ethylbenzene hydroperoxide feed had a temperature of about 92° C. It was found that this temperature could be maintained for more than a month.

1. A process for the preparation of alkylene oxide which process comprises:
(a) oxidizing an organic compound to obtain a hydroperoxide containing stream;
(b) washing the hydroperoxide stream with a basic aqueous solution;
(c) washing the hydroperoxide stream of step (b) with water;
(d) optionally subjecting the hydroperoxide stream obtained in step (c) to distillation;
(e) contacting at least part of the hydroperoxide stream obtained in step (c) and/or (d) with an alkene and a heterogeneous catalyst to obtain a reaction mixture containing a hydroxyl containing compound and alkylene oxide; and,
(f) separating at least part of the alkylene oxide from the reaction mixture, in which process the alkene added in step (e) has a temperature of from 60° C. to 120° C. and the temperature of the hydroperoxide stream contacted with the alkene is similar to the temperature of the hydroperoxide stream obtained in step (c) and/or (d).

2. The process of claim 1, wherein the alkene is propene and the alkylene oxide is propylene oxide.

3. The process of claim 2, wherein the heterogeneous epoxidation catalyst is a titanium containing catalyst.

4. The process of claim 2, comprising heating the alkene in a heat exchanger before contacting with hydroperoxide in step (e).

5. The process of claim 2, wherein the temperature of the alkene added in step (e) is from 70° C. to 110° C. before contacting the hydroperoxide stream.

6. The process of claim 2, wherein the temperature of the hydroperoxide stream is from 80° C. to 110° C. before contacting the alkene.

7. The process of claim 2, wherein step (f) comprises separating the alkylene oxide from the reaction mixture by distillation.

8. The process of claim 1, wherein the heterogeneous epoxidation catalyst is a titanium containing catalyst.

9. The process of claim 1, comprising heating the alkene in a heat exchanger before contacting with hydroperoxide in step (e).

10. The process of claim 1, wherein the temperature of the alkene added in step (e) is from 70° C. to 110° C. before contacting the hydroperoxide stream.

11. The process of claim 1, wherein the temperature of the hydroperoxide stream is from 80° C. to 110° C. before contacting the alkene.

12. The process of claim 1, wherein step (f) comprises separating the alkylene oxide from the reaction mixture by distillation.
